(19) United States
(12) Patent Application Publication
(10) Pub. No.: US 2017/0311933 A1
(43) Pub. Date: Nov. 2, 2017

(54) MRI GUIDED BIOPSY TARGETING SET WITH FIRING OBTURATOR

(71) Applicant: Devicor Medical Products, Inc., Cincinnati, OH (US)

(72) Inventors: Emmanuel V. Tanghal, Mason, OH (US); Kevin Talbot, North Bend, OH (US); Andrew P. Nock, Dayton, OH (US); Robert M. Householder, Loveland, OH (US)

(21) Appl. No.: 15/581,210

(22) Filed: Apr. 28, 2017

Related U.S. Application Data
(60) Provisional application No. 62/329,305, filed on Apr. 29, 2016.

(51) Int. Cl. A61B 10/02; A61B 17/34; A61B 10/00; A61B 90/00

(52) U.S. Cl. CPC A61B 10/0233; A61B 17/3417; A61B 10/0041; A61B 2090/033; A61B 17/3496

(57) ABSTRACT

An MRI guided breast biopsy targeting set includes a firing obturator for use with positioning a breast biopsy device within a patient. The obturator is insertable into a targeting cannula in lieu of a needle of a biopsy device. The targeting set includes an obturator and a firing assembly. The firing assembly includes a housing, a latch mechanism, and a release mechanism, The firing assembly is responsive to the release mechanism to fire the obturator into the desired position within the breast.

### PRIORITY

The present application claims priority to U.S. Provisional Patent Application No. 62/329,305, entitled “MRI Guided Biopsy Targeting Set with Firing Obturator,” filed on Apr. 29, 2016, the disclosure of which is hereby incorporated by reference in its entirety.

### BACKGROUND

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, Positron Emission Mammography (“PEM” guidance), Breast-Specific Gamma Imaging (“BSGI”) guidance or otherwise.

The state of the art technology for conducting a breast biopsy is to use a vacuum-assisted breast biopsy device. A current textbook in this area is “Vacuum-Assisted Breast Biopsy with Mammotome®”, available Nov. 11, 2012, copyright 2013 by Devicor Medical Germany GmBh, published in Germany by Springer Medizin Verlag, Authors: Markus Hahn, Anne Tardivon and Jan Casselman, ISBN 978-3-642-34270-7.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No, 5,928,164, entitled “Apparatus for Automated Biopsy and Collection of Soft Tissue,” issued Jul. 27, 1999; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,626,849, entitled “MRI Compatible Surgical Biopsy Device,” issued Sep. 11, 2003; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,648,466, entitled “Manually Rotatable Piercer,” issued Jan. 19, 2010; U.S. Pat. No. 7,837,632, entitled “Biopsy Device Tissue Port Adjustment,” issued Nov. 23, 2010; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued. Feb. 1, 2012; U.S. Pat. No. 8,206,316, entitled “Tetherless Biopsy Device with Reusable Portion,” issued on Jun. 26, 2012; U.S. Pat. No. 8,241,226, entitled “Biopsy Device with Rotatable Tissue Sample Holder,” issued on Aug. 14, 2012; U.S. Pat. No. 8,251,916, entitled “Revolving Tissue Sample Holder for Biopsy Device,” issued Aug. 28, 2012; U.S. Pat. No. 8,454,531, entitled “Icon-Based User Interface on Biopsy System Control Module,” published May 21, 2009, issued on Jun. 4, 2013; U.S. Pat. No. 8,532,747, entitled “Biopsy Marker Delivery Device,” issued Sep. 10, 2013; U.S. Pat. No. 8,702,623, entitled “Biopsy Device with Discrete Tissue Chambers,” issued on Apr. 22, 2014; U.S. Pat. No. 8,764,680, entitled “Handheld Biopsy Device with Needle Firing,” issued on Jun. 11, 2014; U.S. Pat. No. 8,801,742, entitled “Needle Assembly and Blade Assembly for Biopsy Device,” issued Aug. 12, 2014; U.S. Pat. No. 8,858,465, entitled “Biopsy Device with Motorized Needle Firing,” issued Oct. 14, 2014; U.S. Pat. No. 8,938,285, entitled “Access Chamber and Markers for Biopsy Device,” issued Jan. 20, 2015; U.S. Pat. No. 9,095,326, entitled “Biopsy System with Vacuum Control Module,” issued Aug. 4, 2015 and U.S. Pat. No. 9,095,326, entitled “Biopsy System with Vacuum Control Module,” issued Aug. 4, 2015. The disclosure of each of the above-cited. U.S. Patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006 and now abandoned; U.S. Pat. Pub. No. 2008/0214955, entitled “Presentation of Biopsy Sample by Biopsy Device,” published Sep. 4, 2008; U.S. Pat. Pub. No. 2009/0131821, entitled “Graphical User Interface For Biopsy System Control Module,” published May 21, 2009, now abandoned; U.S. Pat. Pub. No. 2010/0152610, entitled “Hand Actuated Tetherless Biopsy Device with Pistol Grip,” published Jun. 17, 2010, now abandoned; U.S. Pat. Pub. No. 2010/0160819, entitled “Biopsy Device with Central Thumbwheel,” published Jun. 24, 2010, now abandoned; U.S. Pat. Pub. No. 2013/0053724, entitled “Biopsy Device Tissue Sample Holder with Bulk Chamber and Pathology Chamber,” published Feb. 28, 2013, will issue on May 3, 2016 as U.S. Pat. No. 9,326,755; U.S. Pat. Pub. No. 2013/0144188, entitled “Biopsy Device With Slide-in Probe,” published Jun. 6, 2013; and U.S. Pat. Pub. No. 2013/0324882, entitled “Control for Biopsy Device,” published Dec. 5, 2013. The disclosure of each of the above-cited U.S. Patent Application Publications, U.S. Non-Provisional Patent Applications, and U.S. Provisional Patent Applications is incorporated by reference herein.

In U.S. Pat. No. 7,831,290, issued Oct. 20, 2010, the disclosure of which is incorporated by reference herein, a localization mechanism, or fixture, is described that is used in conjunction with a breast coil for breast compression and for guiding a core biopsy instrument during prone biopsy procedures in both open and dosed Magnetic Resonance Imaging (MRI) machines. The localization fixture includes a three-dimensional Cartesian positionable guide for supporting and orienting an MRI-compatible biopsy instrument, and, in particular, a cannula/sleeve to a biopsy site of suspicious tissues or lesions. Another merely illustrative localization mechanism used for guiding a core biopsy instrument is disclosed in U.S. Pat. No. 7,507,210, entitled “Biopsy Cannula Adjustable Depth Stop,” issued Mar. 24, 2009, the disclosure of which is incorporated by reference herein. The localization mechanism includes a grid plate configured to removably receive a guide cube capable of supporting and orienting an MRI-compatible biopsy instrument. For instance, a combination of an obturator and targeting cannula/sleeve may be introduced through a breast to a biopsy site via the guide cube, with proper positioning confirmed using MRI imaging. The obturator may then be removed and the needle of a biopsy device may then be inserted through the targeting cannula/sleeve to reach the targeted lesion.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary targeting set for firing the obturator into position in the breast for use with an MRI breast biopsy system;

FIG. 2 depicts a top plan view of an obturator and obturator actuation assembly of the targeting set of FIG. 1, with a portion of a housing of the obturator actuation assembly removed;

FIG. 3A depicts a top plan view of the obturator actuation assembly of FIG. 1, with the obturator in an initial position;

FIG. 3B depicts another top plan view of the obturator actuation assembly of FIG. 1, with the obturator in a cocked position;

FIG. 3C depicts still another top plan view of the obturator actuation assembly of FIG. 2, with the obturator in a partially fired position;

FIG. 4A depicts a perspective view of the targeting set of FIG. 1, with the obturator in the cocked position;

FIG. 4B depicts another perspective view of the target set of FIG. 1, with the obturator in the partially fired position;

FIG. 5 depicts a perspective view of the targeting set of FIG. 1, with the obturator actuation assembly equipped with an actuation ring;

FIG. 6 depicts a perspective view of an exemplary alternative obturator that may be readily incorporated into the targeting set of FIG. 1;

FIG. 7 depicts a perspective view of another exemplary alternative obturator that may be readily incorporated into the targeting set of FIG. 1; and

FIG. 8 depicts a perspective view of still another exemplary alternative obturator that may be readily incorporated into the targeting set of FIG. 1.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

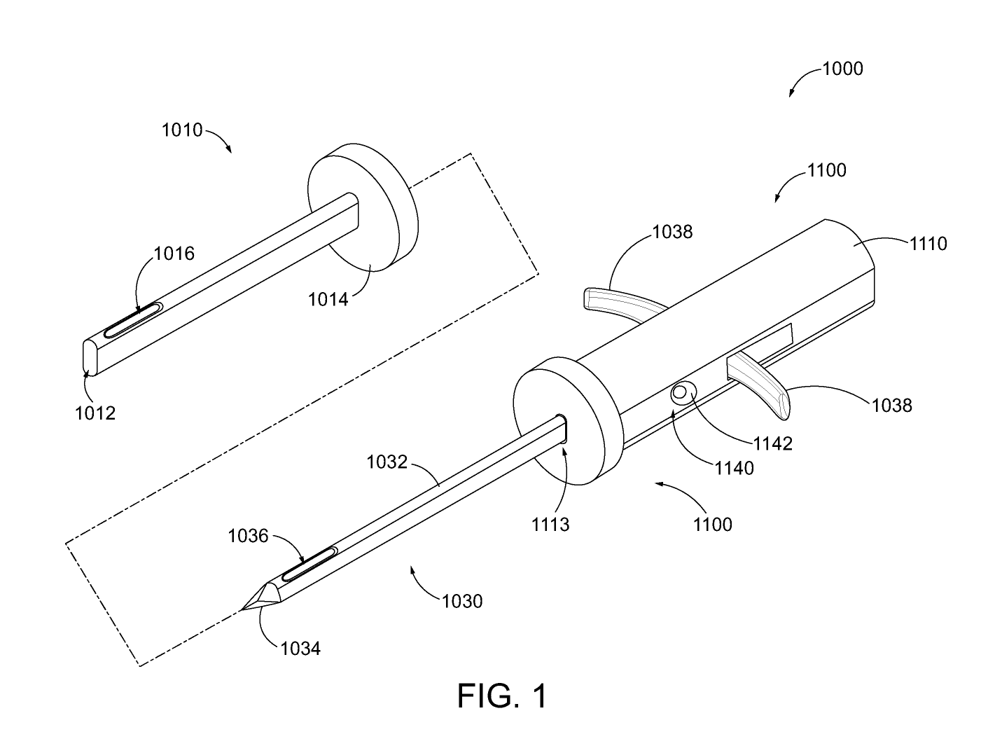

FIGS. 1, 2, 3, 4, 5, 6, 7, 8 and 9 of U.S. Pat. No. 7,507,210, incorporated by reference in its entirety, depict a perspective view of a biopsy system including a control module remotely coupled to a biopsy device, and including a localization fixture with a lateral grid plate used in conjunction with a rotatable cube to position an obturator or a probe of the biopsy device to a desired insertion depth as set by a ring stop.

During the breast biopsy procedure, typically the patient's breasts hang pendulously respectively into breast apertures on the examination table. For convenience, herein a convention is used for locating a suspicious lesion by Cartesian coordinates within breast tissue referenced to localization fixture and to thereafter selectively position an instrument, such as needle of probe that is engaged to holster portion to form biopsy device.

To enhance hands-off use of biopsy system, especially for repeated re-imaging within the narrow confines of a closed bore MRI machine, biopsy system may also guide obturator encompassed by cannula. Depth of insertion is controlled by a depth stop device longitudinally positioned on either needle or cannula. Alternatively, depth of insertion may be controlled in any other suitable fashion.

In typical MRI breast biopsy procedures, a targeting set comprising cannula and obturator is associated with probe. In particular the obturator is slid into cannula and the combination is guided through guide cube to the biopsy site within the breast tissue. The obturator is then withdrawn from cannula, then the needle of the probe is inserted in cannula, and then biopsy device is operated to acquire one or more tissue samples from the breast via needle.

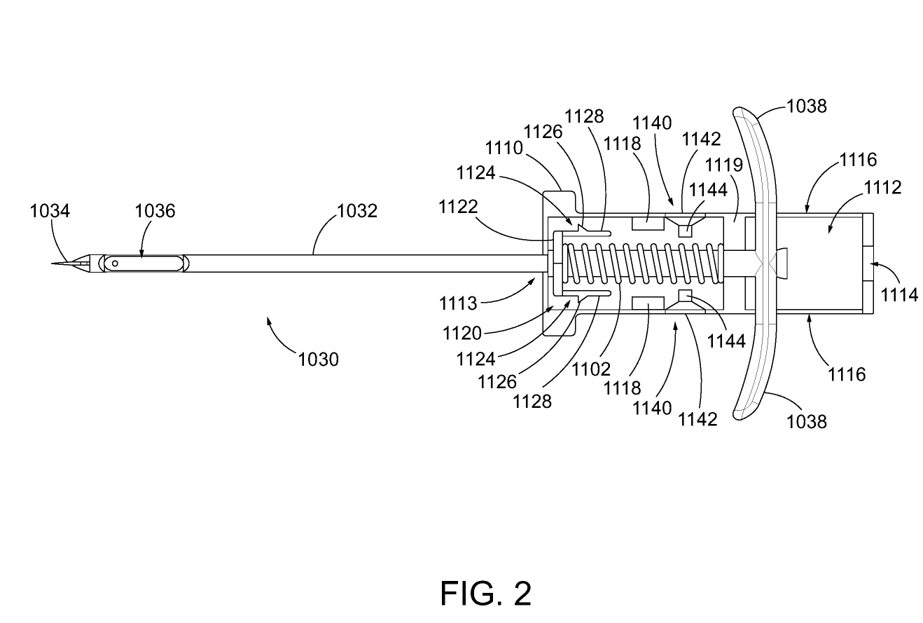

FIGS. 1 and 2 show an exemplary alternative targeting set (1000) for use in association with a probe as similarly described above with respect to targeting set. Like with targeting set, targeting set (1000) of the present example comprises a cannula (1010) and an obturator (1030). However, unlike targeting set described above, targeting set (1000) of the present example includes an obturator actuation assembly (1100). As will be described in greater detail below, targeting set (1000) is generally configured such that obturator (1030) is independently actuatable relative a cannula (1010) while cannula (1010) remains longitudinally fixed via a depth stop member (not shown). Although targeting set (1000) is described herein as being usable with cannula (1010), it should be understood that cannula (1010) in the present example may be omitted entirely in some examples. In such examples, obturator (1010) and components associated with obturator (1010) generally operate in substantially the same way unless otherwise noted herein. In still other examples, cannula (1010) can alternatively be replaced with cannula (94) described above.

Cannula (1010) of the present example defines a lumen (not shown) and includes an open distal end (1012) and a lateral aperture (1016). Lateral aperture (1016) is in communication with the lumen defined by cannula (1010). As will be described in greater detail below, lateral aperture (1016) is configured to receive tissue such that tissue can pass through cannula (1010) and into either obturator (1030) or needle (90) of biopsy device (14). The proximal end of cannula (1010) is fixedly secured to a hub (1014). Although not shown, it should be understood that in some examples hub (1014) includes attachment features (not shown) and/or ports (not shown). In such examples, the attachment features can facilitate coupling of hub (1014) to a portion of obturator actuation assembly (1100), while the ports can permit communication of fluids with the lumen defined by cannula (1010). In examples that include ports, such ports may be optionally coupled to a fluid source for delivery of therapeutic substances, saline, or other fluids to a biopsy site via the lumen. While also not shown, it should be understood that hub (1014) includes other features and/or components such as seals, thumbwheels, fluid channels, and/or additional lumen similar to cylindrical hub described above.

Obturator (1030) of the present example comprises a rigid elongate shaft (1032) having a sharp distal tip (1034) and an oval-shaped transverse cross-section. Shaft (1032) of the present example comprises a single MRI compatible material such as ceramic or plastic, although no such limitation is intended. For instance, in other examples shaft (1032) comprises a non-MRI compatible material such as metal. However, in such examples, obturator (1030) may be removed from cannula (1010) during an MRI imaging procedure.

Shaft (1032) further includes a lateral aperture (1036) oriented proximally of the sharp distal tip (1034). Lateral aperture (1036) is positioned on shaft (1032) to align with lateral aperture (1016) of cannula (1010) when obturator (1030) is fully inserted into cannula (1010). Lateral aperture (1036) extends laterally into shaft (1032) to thereby form a chamber, notch, or recess in shaft (1032). As will be described in greater detail below, this configuration permits lateral aperture (1036) of obturator (1030) to receive tissue therein to thereby provide MRI visualization of the position of lateral apertures (1016, 1036) when obturator (1030) and cannula (1010) are disposed within a patient. Although not shown, it should be understood that in some examples shaft (1032) may include one or more lumens extending from lateral aperture (1036) to the proximal end of shaft (1032). When shaft (1032) is equipped with such lumens, the lumens can permit an operator to communicate fluid to or from lateral aperture (1036). Of course, such lumens are merely optional and in some examples shaft (1032) is merely solid with the exception of the chamber, recess, or notch formed by lateral aperture (1036). In addition, it should be understood that in some examples lateral aperture (1036) can be omitted entirely and shaft (1036) can therefore be completely solid. In such examples, obturator (1030) is used only to penetrate tissue, while a separate imaging element may be used in place of obturator (1030) once obturator (1030) is removed from cannula (1010). This separate imaging element can then be used to aid in visualizing lateral aperture (1016) of cannula (1010).

FIG. 1 shows obturator (1030) and obturator actuation assembly (1100). As can be seen, obturator actuation assembly (1100) generally comprises an outer housing (1110), a resilient member (1102), a latch mechanism (1120), and a release mechanism (1140). As will be described in greater detail below, latch mechanism (1120) is generally selectively movable relative to outer housing (1110) to load resilient member (1102) with potential energy such that release mechanism (1140) can be used to fire obturator (1030) distally 1 to 3 cm. Such a firing action permits sharp tip (1034) of obturator (1030) to rapidly cut through and penetrate tissue without generally displacing said tissue. As will be understood, the firing distance for obturator (1030) is generally predetermined such that it is fixed and cannot be varied by an operator. However, in some examples certain features may be added to permit selective adjustment of the firing distance for obturator (1030) by an operator.

In FIG. 2, the entire length of obturator (1030) is visible. As can be seen, obturator (1030) includes a pair of laterally extending arms (1038) oriented at the proximal end of shaft (1032). Arms (1038) are generally symmetrical and extend outwardly from shaft (1032) of obturator (1030). Each arm (1038) bows distally as each arm extends outwardly. As will be understood, arms (1038) are generally configured to be manipulated by an operator to pull obturator (1030) proximally relative to outer housing (1110) of obturator actuation assembly (1100). Accordingly, it should be understood that the bowed configuration of arms (1038) may assist an operator in gripping arms (1038). In other examples, arms (1038) can include numerous alternative shapes that may increase or decrease operator grip. Thus, it should be understood that the bowed configuration of arms (1038) is merely optional and may be omitted in some examples.

Outer housing (1110) of obturator actuation assembly (1100) comprises a generally cylindrical shell that defines a generally hollow interior (1112), a pair of channels (1116), a pair of lock catches (1118), and an internal wall (1119). Hollow interior (1112) is in communication with the exterior of outer housing (1110) through a distal opening (1113) and a proximal opening (1114). Distal opening (1113) is sized to receive shaft (1032) of obturator (1030) such that shaft (1032) can pass through outer housing (1110) and into hollow interior (1112). Proximal opening (1114) is positioned on the proximal end of outer housing (1110) and is configured to align with the proximal end of obturator (1100). In the present example, proximal opening (1114) can be used to provide visual confirmation of the positioning of obturator (1030) relative to outer housing (1110). For instance, as will be described in greater detail below, the proximal end of obturator (1030) is generally movable toward proximal opening (1114). As the proximal end of obturator (1030) is moved closer to proximal opening (1114), at least a portion of the proximal end may become visible to an operator. Additionally, in examples where obturator (1100) includes one or more lumens, proximal opening (1114) can be usable to permit tube, conduits, or other fluid handing devices to communicate with obturator (1030) through outer housing (1110).

Each channel (1116) of outer housing (1110) includes a generally rectangular opening defined in outer housing (1110) on opposite sides of outer housing (1110). As can be seen, each channel (1116) is configured to permit a corresponding arm (1038) of obturator (1030) to extend outwardly of outer housing (1110). This feature makes arms (1038) accessible to an operator from the exterior of outer housing (1110). Each channel (1116) is further configured to be longitudinally elongate. As will be described in greater detail below, this feature permits each arm (1038) of obturator (1030) to actuate proximally and distally relative to outer housing (1110) so that obturator (1030) may actuate between a cocked position and an initial or fired position.

Two lock catches (1118) protrude inwardly into hollow interior (1112) of outer housing (1110) from the exterior of outer housing (1110). Lock catches (1118) comprise rectangular shaped protrusions. As will be described in greater detail below, each lock catch (1118) is configured to engage a corresponding portion of latch mechanism (1120) to permit latch mechanism (1120) to selectively lock obturator (1030) in the cocked position. Latch mechanism (1120) may then be released from engagement with lock catches (1118) using release mechanism (1140). Although lock catches (1118) are shown as having a primarily rectangular shape, it should be understood that in other examples numerous alternative shapes can be used such as triangular, ovular, irregular, a combination of a plurality of different shapes, or any other shape as will be apparent to those of ordinary skill in the art in view of the teachings herein.

Internal wall (1119) is disposed within hollow interior (1112) of outer housing (1110) and extends laterally across hollow interior (1112). Internal wall (1119) is disposed distally of each channel (1116). This positioning of inner wall (1119) permits inner wall (1119) to act as a mechanical ground for resilient member (1102). Additionally, internal wall (1119) includes an opening (not shown) extending therethrough such that obturator (1030) can pass though internal wall (1119). As will be described in greater detail below, this configuration permits movement of obturator (1030) relative to internal wall (1119), while preventing movement of resilient member (1102) proximally past internal wall (1119). As will be understood, this permits resilient member (1102) to build up potential energy while obturator (1030) is moved proximally relative to internal wall (1119).

The opening is internal wall (1119) further serves the function of maintaining axial alignment of obturator (1030) within outer housing (1110). For instance, as obturator (1030) moves longitudinally through the opening in inner wall (1119), lateral displacement of obturator (1030) is generally resisted. Similarly, although not shown, it should be understood that in some examples outer housing (1110) may include other structures such as additionally walls or bosses to further assist with maintaining the axial alignment of obturator (1030) within outer housing (1110). Of course, such structures are entirely optional and may be omitted in some examples.

Latch mechanism (1120) is disposed entirely within outer housing (1110). As described above, latch mechanism (1120) is configured to engage lock catches (1118) of outer housing (1110) to selectively lock obturator (1030) in the cocked position and to be released by actuation of release mechanism (1140). Latch mechanism (1120) comprises a base (1122) and two proximally extending latch arms (1124). Base (1120) of the present example is fixedly secured to obturator (1030). In some examples, obturator (1030) may include an annular flange, a recess, and/or other geometric feature to aid in securing base (1120) to obturator (1030). In still other examples, base (1120) may be unitary with obturator (1030) such that base (1120) and obturator (1030) together form a single part. Regardless, base (1120) extends outwardly from obturator (1030) to support latch arms (1124).

Each latch arm (1124) extends proximally from base (1122). Latch arms (1124) of the present example are shown as being unitary with base (1122) such that base (1122) and latch arms (1124) form a single unitary part. However, it should be understood that in other examples base (1122) and latch arms (1124) can be separate components. Regardless of the particular construction of base (1122) and latch arms (1124), it should be understood that latch arms (1124) of the present example comprise a generally resilient material such that each latch arm (1124) is generally rigid, yet deformable. This material property permits each latch arm (1124) to selectively engage and disengage with each respective lock catch (1118) of outer housing (1110).

Each latch arm (1124) defines a latch portion (1126) and a release portion (1128). Each latch portion (1126) extends outwardly from its respective latch arm (1124) and defines a generally triangular shape. This triangular shape forms a right triangle with the hypotenuse oriented proximally and one leg oriented distally. This orientation of each latch portion (1126) permits the hypotenuse to act as a ramp and the distally facing leg to act as a catch. Thus, the hypotenuse acts to deflect each latch arm (1124) away from a respective lock catch (1118) as obturator (1030) is retracted proximally, before the distally oriented leg is forced into engagement with the proximal side of the respective lock catch (1118).

Each release portion (1128) extends proximally of a given latch portion (1126). The positioning of each release portion (1128) is such that each release portion (1128) is laterally spaced away from a respective lock catch (1118). As will be described in greater detail below, this permits each release portion (1128) to pass unobstructed beyond the respective lock catch (1118) to engage with release mechanism (1140) as obturator (1030) is retracted proximally. As will also be described in greater detail below, each release portion (1128) is configured to be acted upon by release mechanism (1140) to deflect each latch arm (1124) inwardly and thereby disengage latch portions (1126) from lock catches (1118).

Release mechanism is positioned proximally of each lock catch (1118) of outer housing (1110). Release mechanism comprises two buttons (1142) extending through outer housing (1110) on either side of outer housing (1110). Each button (1142) includes an inwardly directed protrusion (1144) that extends into hollow interior (1112) of outer housing (1110). In the present example, buttons (1142) and each corresponding protrusion are unitarily movable relative to outer housing (1110). In some examples, this movability can be facilitated by a rubber gasket or resilient member connecting each button (1142) to outer housing (1110) to permit some movement, yet ultimately keep each button (1142) attached to outer housing (1110). In other examples, this movability is facilitated by each button (1142) being configured to be flexible to permit movement of buttons (1142) and protrusions (1144) outwardly. Regardless, it should be understood that each button (1142) and each corresponding protrusion (1144) is movable to permit each button (1142) to selectively engage each respective latch arm (1142). As will be described in greater detail below, when each latch arm (1124) is engaged with lock catch (1118), an operator may actuate each button (1142) to force each protrusion (1144) into engagement with release portion (1128) of each latch arm (1124) to thereby disengage each latch arm (1124) from a corresponding lock catch (1118).

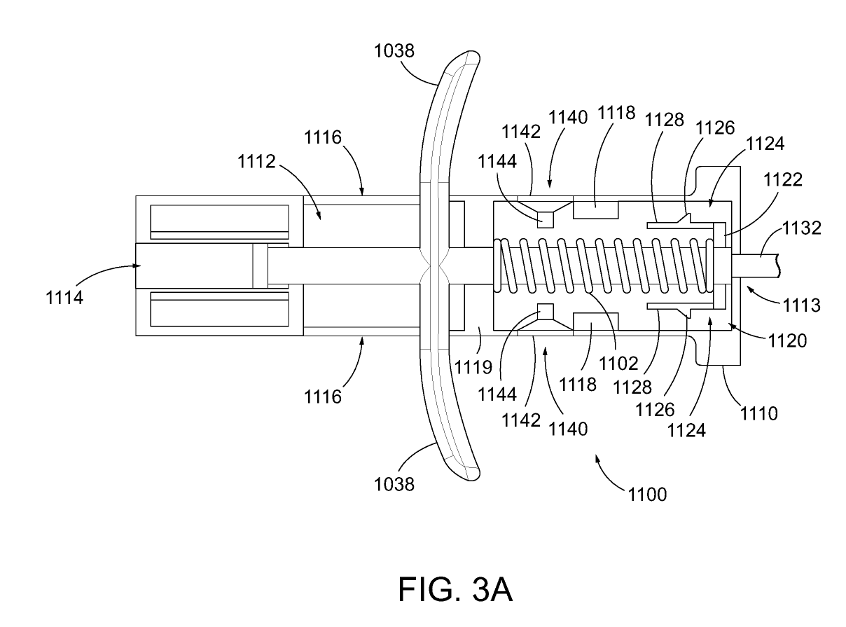

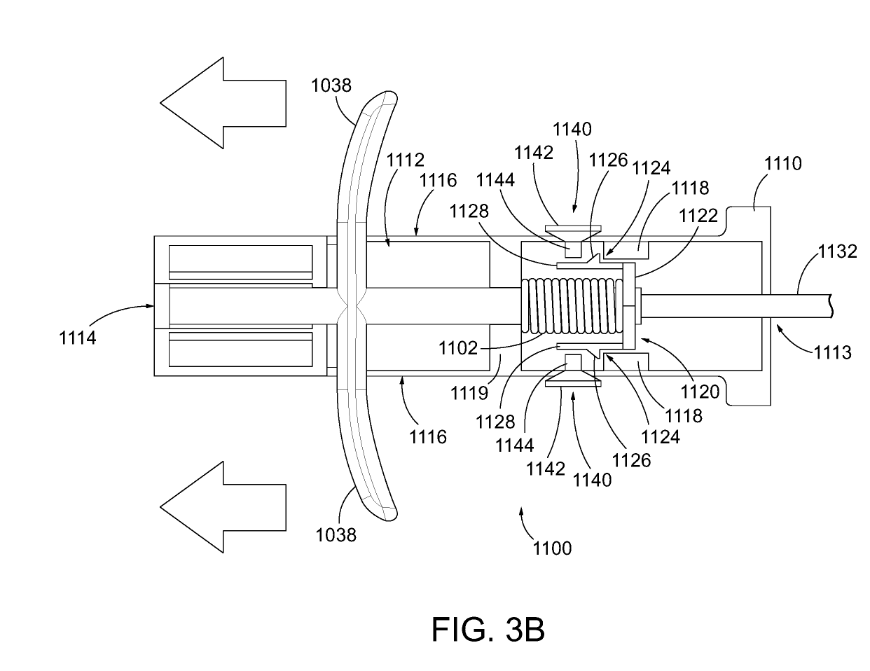

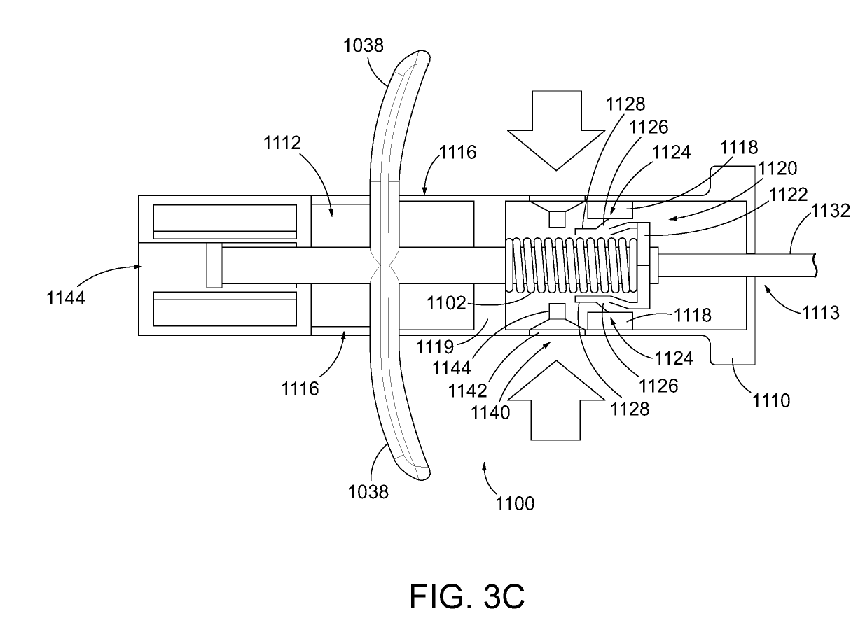

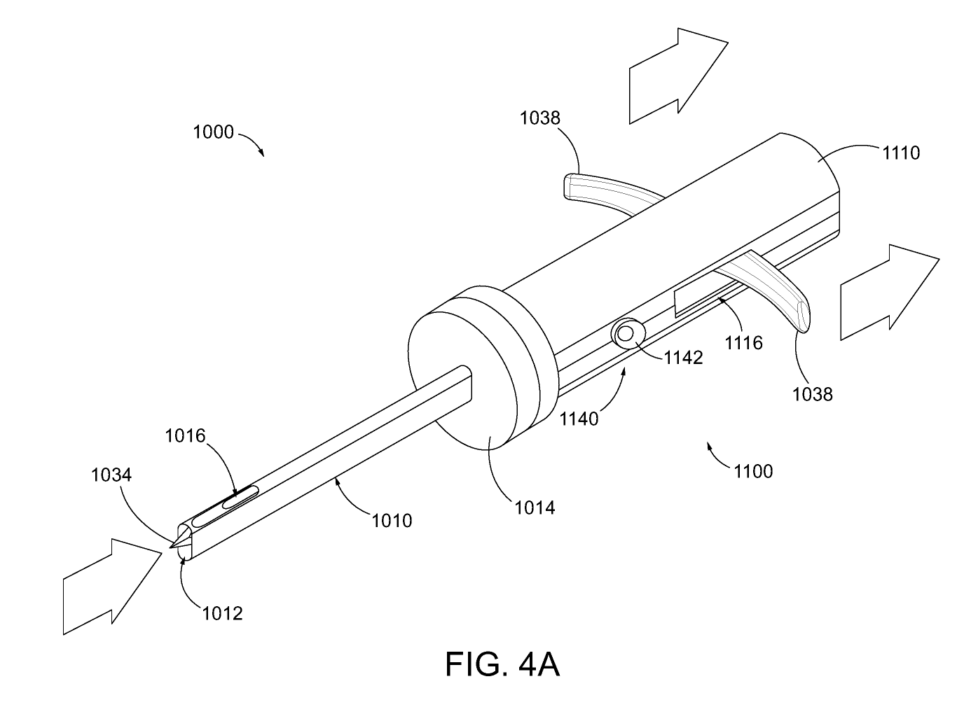

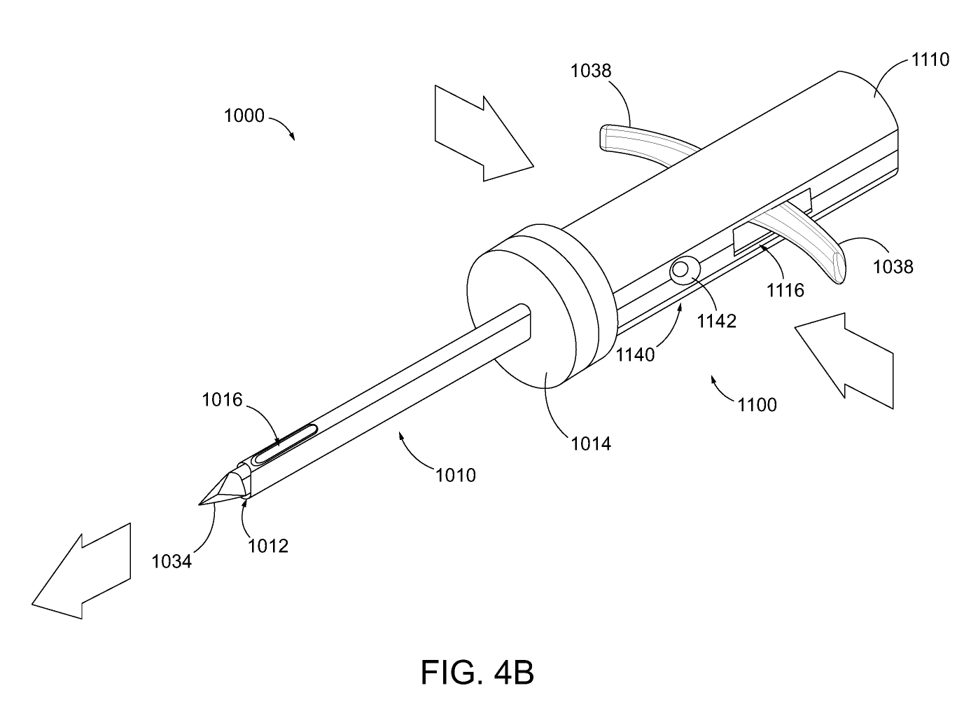

An exemplary use of targeting set (1000) is shown in FIGS. 3A-4B. As can be seen in FIG. 3A, obturator (1030) initially begins in an initial position. In the initial position, obturator (1030) is advanced to its furthest distal position. In this position, sharp tip (1034) of obturator (1030) protrudes from the open distal end (1012) of cannula (1010) a distance of approximately 3 cm. With sharp tip (1034) protruding from open distal end (1012), an operator may use obturator to initially penetrate tissue by grasping arms (1038) of obturator (1030) and pushing obturator (1030) and cannula (1010) through guide cube (104) and into tissue of a patient. However, to avoid “snowplowing” or “tenting” of tissue at the target site, an operator may cease advancement of obturator (1030) about two or more centimeters prior to reaching the target site.

Once the obturator (1030) and cannula (1010) are positioned near the target site within the patient, an operator may desire to rapidly fire the obturator (1030) to drive sharp tip (1034) through the patient's tissue, thereby avoiding “snowplowing” or “tenting” of tissue. To initiate the firing sequence, an operator will begin by moving the obturator (1030) to the cocked position as shown in FIGS. 3B and 4A. To move obturator (1030) to the cocked position, an operator will grasp arms (1038) of obturator (1030) and pull obturator (1030) proximally via arms (1038). Obturator (1030) will move proximally until it reaches its furthest proximal position relative to outer housing (1110). Alternatively, it should be understood that in examples where cannula (1010) is omitted, obturator (1030) may instead be moved to the cocked position prior to insertion into the patient. In other words, in examples where cannula (1010) is omitted an operator may first cock obturator (1030) by pulling arms (1038) proximally and then proceed to insert obturator (1030) into a patient, positioning obturator (1030) adjacent to the target site.

As obturator (1030) is pulled proximally, latch mechanism (1120) is simultaneously retracted proximally a corresponding distance. In particular, because base (1122) of latch mechanism (1120) is fixedly secured to obturator (1030), base (1122) and latch arms (1124) will move proximally with obturator (1030). Once latch portion (1126) of each latch arm (1124) contacts a respective lock catch (1118) of outer housing (1110), the hypotenuse of the triangular latch portion (1126) will operate to deform a given latch arm (1124). Each latch portion (1126) will then move along the interior of each lock catch (1118) until each latch portion (1126) is pulled past each lock catch (1118). This will permit each latch arm (1124) to resiliently return to its initial non-deformed state and thereby engage the proximal end of each lock catch via the leg of each latch portion (1126). Obturator (1030) is then secured in the cocked position through this engagement between latch arms (1124) and lock catches (1118).

Also during proximal movement of obturator (1030), resilient member (1102) is compressed between internal wall (1119) of outer housing (1110) and base (1122) of latch mechanism (1120) to thereby store potential energy in resilient member (1102). In the present example, resilient member (1102) comprises a coil spring positioned co-axially with obturator (1030). Of course, in other examples any other suitable resilient feature may be used in place of resilient member (1102) such as leaf springs, compressive rubber, and/or etc. Once obturator (1030) is positioned in the cocked position, resilient member (1102) is fully compressed and is therefore positioned to drive obturator (1030) distally relative to outer housing (1110) via base (1122) of latch mechanism (1120).

As can best be seen in FIG. 4A, as obturator (1030) is retracted, sharp tip (1034) moves proximally relative to open distal end (1012) of cannula (1010). Thus, when obturator (1030) is moved to the cocked position, sharp tip (1034) is retracted at least partially within cannula (1010). Once sharp tip (1034) is retracted within cannula (1010), cannula (1010) may be advanced further by an operator to the target position. When advanced, sharp tip (1034) of obturator (1030) is retracted, so further engagement of tissue does not occur. However, it should be understood that once obturator (1030) is fired, obturator (1030) will be positioned at the target site. It should be understood that in examples where cannula (1010) is omitted, such a repositioning step is not necessary and obturator (1030) may be immediately fired to position lateral aperture (1036) of obturator (1030) at the target site. Indeed, in certain contexts, using obturator (1030) without cannula (1010) may be desirable to minimize disturbance of tissue at or near the target site prior to firing of obturator (1030).

Once obturator (1030) has been positioned near the target site and cocked as described above, an operator may next initiate firing of obturator (1030). To initiate firing, an operator merely presses both buttons (1142) of release mechanism (1140) on either side of outer housing (1110). As is best seen in FIG. 3C, this causes protrusions (1144) of each button (1142) to push release portion (1128) of each latch arm (1124) inwardly. This in turn causes latch arms (1124) to deflect inwardly and disengage from lock catches (1118) of outer housing (1110). It should be understood that due to the present configuration, both buttons (1142) must be pressed simultaneously to initiate firing. In the present example, this feature may be desirable to avoid inadvertent firing of obturator (1030). However, it should be understood than in other examples, obturator actuation assembly (1100) may be reconfigured for use with only a single button (1142), or may alternatively be configured with multiple buttons (1142) but only depression of a single button (1142) being required for tiring.

Once latch arms (1124) of latch mechanism (1120) have been disengaged from lock catches (1118) of outer housing (1110), obturator (1030) is free to move distally. With obturator (1030) free, the potential energy built up in resilient member (1102) during cocking will release and rapidly drive obturator (1030) distally as resilient member (1102) expands. In particular, expansion of resilient member (1102) rapidly pushes base (1122) of latch mechanism (1120) away from internal wall (1119) of outer housing (1110). Because base (1122) is fixedly secured to obturator (1030), distal movement of base (1122) will result in distal movement of obturator (1030). This relatively quick distal motion of obturator (1030) will force sharp tip (1034) of obturator (1030) approximately 2 cm through tissue while substantially reducing displacement of said tissue relative to sharp tip (1034). As can be seen in FIG. 3B, in examples where obturator (1030) is used with cannula (1010), this will result in sharp tip (1034) protruding out of open distal end (1012) of cannula (1010) 1 to 3 cm. Of course, in examples where cannula (1010) is omitted, obturator (1030) will merely move through tissue of a patient. Regardless, obturator (1030) will be positioned at the target site and the operator may initiate various steps related to the performance of a biopsy procedure.

In some instances it may be desirable to modify certain feature of obturator (1030) or obturator actuation assembly (1100) described above to promote ease of use. For instance, in some examples it may be desirable to add certain components to obturator actuation assembly (1100) to enhance the ability of an operator to move obturator (1030) to the cocked position. In other examples, it may be desirable to modify obturator (1030) itself. For instance, in some examples the particular shape of arms (1038) may be modified to enhance the ability of an operator to grip obturator (1030). Various modified versions of obturator actuation assembly (1100) and/or obturator (1030) are described below. While specific examples are described herein, it should be understood that various modification may be desirable as will be apparent to those of ordinary skill in the art in view of the teachings herein.

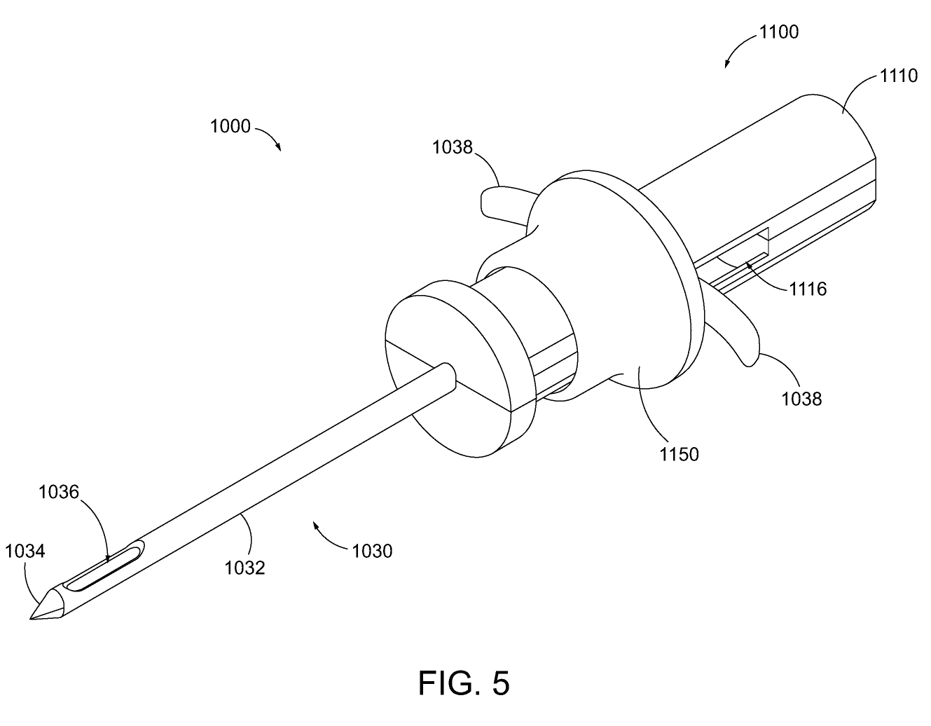

FIG. 5 shows targeting set (1000) described above, except in this examples, outer housing (1110) is equipped with an actuation ring (1150). It should be understood that with the exception of actuation ring (1150), every other component of outer housing (1110) remains the same. Actuation ring (1150) is configured to abut the distal end of arms (1038) of obturator (1030). Actuation ring (1150) is further configured to coaxially encompass the generally cylindrical shape of outer housing (1110). The shape of actuation ring (1150) is generally ring shaped with a progressively expanding outer diameter as actuation ring (1150) extends proximally. This progressively expanding outer diameter of actuation ring (1150) is configured to enhance operator grip from all sides around the exterior of outer housing (1110). Additionally, it should be understood that in the present example the longitudinal extension of actuation ring (1150) is configured to cover buttons (1142) of release mechanism (1140) until obturator (1030) is transitioned into the cocked position. This feature may be desirable to prevent inadvertent depression of buttons (1142) prior to cocking obturator (1130) and to provide an operator with physical feedback to determine whether obturator (1130) is cocked.

In use, obturator actuation assembly (1100) is used as described above when equipped actuation ring (1150). However, when an operator is cocking obturator actuation assembly (1100), actuation ring (1150) is used by an operator to drive obturator (1030) to the cocked position instead of arms (1038). Actuation ring (1150) then acts on arms (1038) of obturator (1030) instead of an operator directly grasping arms (1038). It should be understood that this configuration may in some instances enhance the usability of targeting set (1000). For example, because actuation ring (1150) extends entirely around outer housing (1110) of obturator actuation assembly (1100), actuation ring (1150) is accessible to an operator regardless of the positioning of obturator actuation assembly (1100). Thus in some instances it may be desirable to use an actuation ring (1150) equipped obturator actuation assembly (1100) where access to arms (1038) of obturator (1030) may otherwise be obstructed.

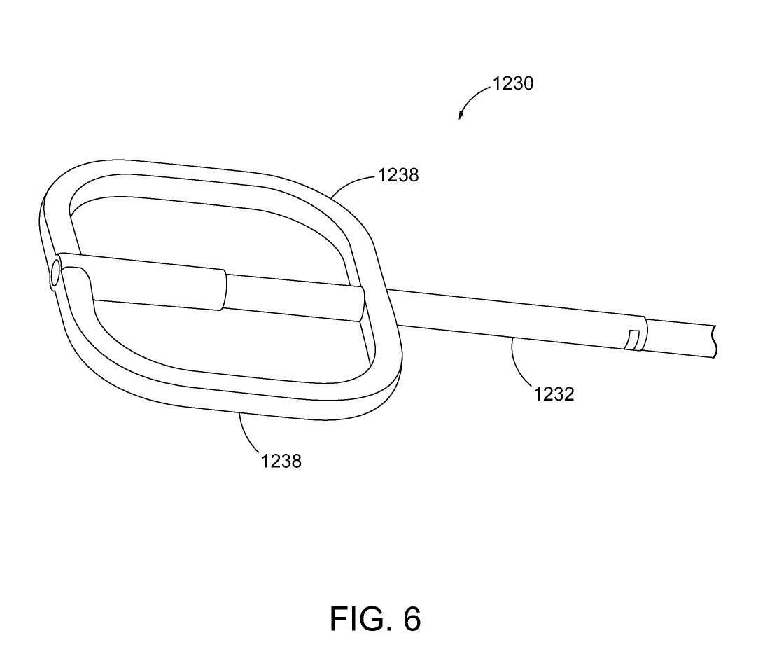

FIG. 6 shows an obturator (1230) with arms (1238) having an alternative configuration relative to arms (1038) of obturator (1030) described above. It should be understood that unless otherwise noted herein, obturator (1230) is substantially the same as obturator (1030) described above. For instance, obturator (1230) of the present example comprises a rigid elongate shaft (1232) having a sharp distal tip (not shown) and an oval-shaped transverse cross-section. However, unlike obturator (1030), obturator (1230) of the present example is equipped with D-shaped arms (1238) rather than bowed arms (1038). The D-shape of arms (1238) is generally configured to enhance an operator's ability to grip arms (1238). Aside from gripping techniques, it should be understood that arms (1238) are used identically as arms (1038) described above.

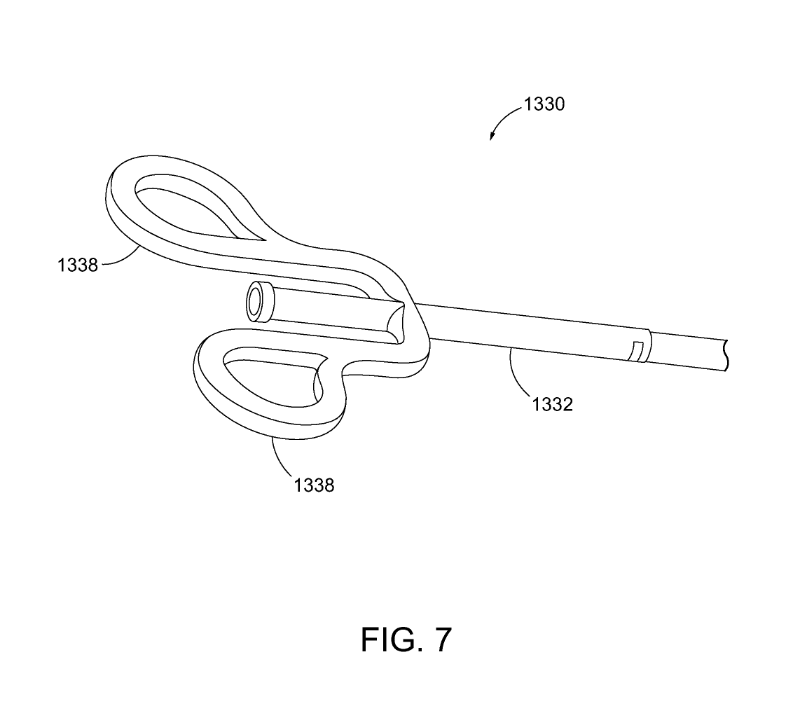

FIG. 7 shows an obturator (1330) with arms (1338) having an alternative configuration relative to arms (1038) of obturator (1030) described above. It should be understood that unless otherwise noted herein, obturator (1330) is substantially the same as obturator (1030) described above. For instance, obturator (1330) of the present example comprises a rigid elongate shaft (1332) having a sharp distal tip (not shown) and an oval-shaped transverse cross-section. However, unlike obturator (1030), obturator (1330) of the present example is equipped with finger ring arms (1338) rather than bowed arms (1038). The addition of finger rings with arms (1338) is generally configured to enhance an operator's ability to grip arms (1338). Aside from gripping techniques, it should be understood that arms (1338) are used identically as arms (1038) described above.

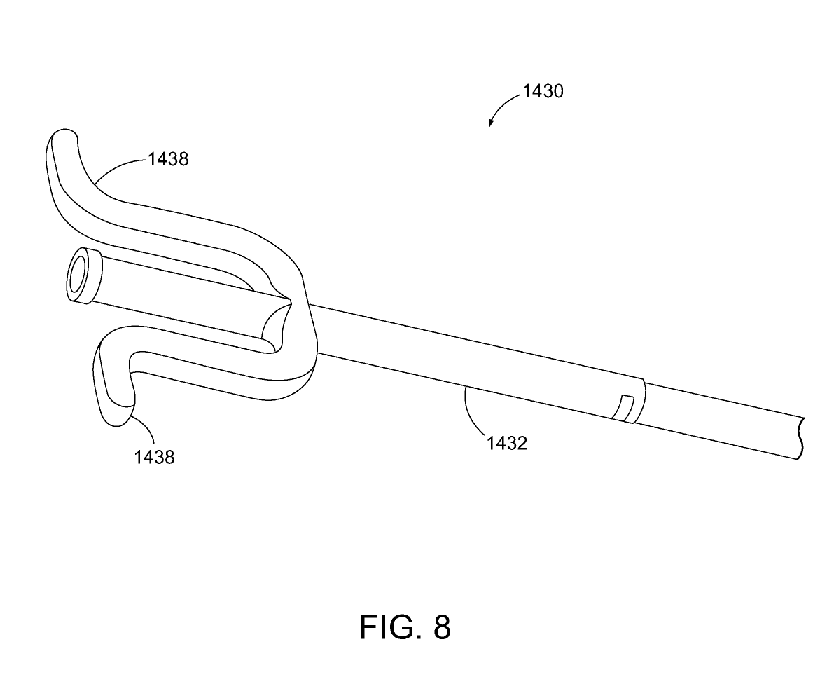

FIG. 8 shows an obturator (1430) with arms (1438) having an alternative configuration relative to arms (1038) of obturator (1030) described above. It should be understood that unless otherwise noted herein, obturator (1430) is substantially the same as obturator (1030) described above. For instance, obturator (1430) of the present example comprises a rigid elongate shaft (1432) having a sharp distal tip (not shown) and an oval-shaped transverse cross-section. However, unlike obturator (1030), obturator (1430) of the present example is equipped with proximally extending arms (1438) rather than bowed arms (1038). In particular, arms (1438) are configured to extend proximally and longitudinally relative to obturator (1420) until approximately the proximal end of obturator (1420). Near the proximal end of obturator (1430), arms (1438) shift direction and extends transversely and outwardly from obturator (1430). This feature of arms (1438) described above is generally configured to enhance an operator's ability to grip arms (1438). Aside from gripping techniques, it should be understood that arms (1438) are used identically as arms (1038) described above.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A targeting set for use with positioning a biopsy device within a patient, the targeting set comprising: (a) an obturator; and (b) a firing assembly, wherein the firing assembly comprises: (i) a housing, (ii) a latch mechanism, and (iii) a release mechanism, wherein the firing assembly is responsive to the release mechanism to disengage the latch mechanism and fire the obturator distally relative to the housing.

### EXAMPLE 2

The targeting set of Example 1, wherein the obturator extends longitudinally through the housing of the firing assembly.

### EXAMPLE 3

The targeting set of Example 2, wherein the obturator comprises a pair of laterally extending arms, wherein the pair of arms are configured to translate the obturator proximally from an initial position to a cocked position.

### EXAMPLE 4

The targeting set of Example 3, wherein translation of the obturator to the cocked position is configured to engage the latch mechanism.

### EXAMPLE 5

The targeting set of Example 4, wherein the latch mechanism is configured to selectively maintain the obturator in the cocked position.

### EXAMPLE 6

The targeting set of any one or more of Examples 3 through 5, wherein the laterally extending arms are bowed distally.

### EXAMPLE 7

The targeting set of any one or more of Examples 3 through 5, wherein each arm of the laterally extending arms define grip rings.

### EXAMPLE 8

The targeting set of any one or more of Examples 1 through 7, further comprising a cannula assembly, the cannula assembly comprising a hub and a cannula extending distally from the hub.

### EXAMPLE 9

The targeting set of Example 8, wherein the cannula defines a lumen extending longitudinally from an open distal tip through the hub, wherein the lumen is configured to receive the obturator.

### EXAMPLE 10

The targeting set of Example 9, wherein the obturator is movable relative to the cannula to retract the obturator within the lumen of the cannula.

### EXAMPLE 11

The targeting set of Example 9, wherein the cannula comprises a first lateral aperture, wherein the obturator comprises a second lateral aperture, wherein the first and second lateral apertures are configured to align to receive tissue therein.

### EXAMPLE 12

The targeting set of any one or more of Examples 1 through 11, wherein the obturator comprises a sharp distal tip.

### EXAMPLE 13

The targeting set of any one or more of Examples 1 through 12, wherein the firing assembly further comprises a resilient member, wherein the resilient member is configured to drive the obturator distally.

### EXAMPLE 14

The targeting set of Example 13, wherein the resilient member comprises a coil spring.

### EXAMPLE 15

The targeting set of Example 14 wherein the coil spring is oriented coaxially with the obturator.

### EXAMPLE 16

A biopsy system, comprising: (a) a biopsy device, wherein the biopsy device comprises: (i) a body, (ii) a needle, and (iii) a cutter, wherein the needle extends from the body to collect tissue samples using the cutter; and (b) a targeting set, wherein the targeting set comprises: (i) a cannula, (ii) an obturator having an elongate shaft and a sharp distal tip, and (iii) an obturator actuation assembly, wherein the cannula defines a lumen, wherein the lumen is configured to separately receive the needle of the biopsy device and the obturator, wherein the obturator actuation assembly is configured to fire the obturator distally relative to the cannula to project the obturator out of the cannula and through tissue.

### EXAMPLE 17

The biopsy system of Example 16, wherein the obturator actuation assembly is configured to drive the obturator between an initial position and a cocked position, wherein the obturator actuation assembly is further configured to selectively maintain the obturator in the cocked position.

### EXAMPLE 18

The biopsy system of Example 16, wherein the obturator actuation assembly comprises a housing, a latch mechanism and a release mechanism, wherein the latch mechanism is secured to the obturator to selectively engage at least a portion of the housing.

### EXAMPLE 19

The biopsy system of Example 18, wherein the release mechanism is configured to selectively disengage the latch mechanism from the housing.

### EXAMPLE 20

A method for placing a targeting set at a biopsy site, wherein the targeting set comprises an obturator and an obturator actuation assembly, wherein the method comprises: (a) drawing the obturator proximally relative to the obturator actuation assembly to place the obturator and the obturator actuation assembly in a cocked configuration; (b) inserting the obturator into tissue of a patient; (c) advancing the obturator to a first position, wherein the first position is within a predetermined distance of the biopsy site; and (d) firing the obturator distally under the direction of the obturator actuation assembly, wherein firing the obturator distally advances the obturator to the biopsy site.

### EXAMPLE 21

An MRI guided breast biopsy targeting set with firing obturator for use with positioning a biopsy device within a patient, the targeting set comprising: (a) an obturator, wherein the obturator is insertable into a targeting cannula in lieu of a needle of a biopsy device; and (b) a firing assembly, wherein the firing assembly includes: (i) a housing, (ii) a latch, and (iii) a release, wherein the firing assembly is responsive to the release to disengage the latch and fire the obturator distally relative to the housing.

### EXAMPLE 22

The targeting set of Example 21, wherein the obturator extends longitudinally through the housing of the firing assembly.

### EXAMPLE 23

The targeting set of Example 22, wherein the obturator comprises a pair of laterally extending arms, wherein the pair of arms are configured to translate the obturator proximally from an initial position to a cocked position.

### EXAMPLE 24

The targeting set of Example 23, wherein translation of the obturator to the cocked position is configured to engage the latch mechanism.

### EXAMPLE 25

The targeting set of Example 24, wherein the latch is configured to selectively maintain the obturator in the cocked position.

### EXAMPLE 26

The targeting set of Example 23, wherein the laterally extending arms are bowed distally.

### EXAMPLE 27

The targeting set of Example 23, wherein the laterally extending arms define grip rings.

### EXAMPLE 28

The targeting set of Example 21, further comprising a cannula assembly, the cannula assembly comprising a hub and a cannula extending distally from the hub.

### EXAMPLE 29

The targeting set of Example 28, wherein the cannula defines a lumen extending longitudinally from an open distal tip through the hub, wherein the lumen is configured to receive the obturator.

### EXAMPLE 30

The targeting set of Example 29, wherein the obturator is movable relative to the cannula to retract the obturator within the lumen of the cannula.

### EXAMPLE 31

The targeting set of Example 29, wherein the cannula comprises a first lateral aperture, wherein the obturator comprises a second lateral aperture, wherein the first and second lateral apertures are configured to align to receive tissue therein when the obturator is in an un-cocked position.

### EXAMPLE 32

The targeting set of Example 21, wherein the obturator comprises a sharp distal tip.

### EXAMPLE 33

The targeting set of Example 21, wherein the firing assembly further comprises a resilient member, wherein the resilient member is configured to drive the obturator distally.

### EXAMPLE 34

The targeting set of Example 33, wherein the resilient member comprises a coil spring.

### EXAMPLE 35

The targeting set of Example 34, wherein the coil spring is oriented coaxially with the obturator.

### EXAMPLE 36

An MRI guided breast biopsy system, comprising: (a) a biopsy device, wherein the biopsy device includes: (i) a body, (ii) a needle, and (iii) a cutter, wherein the needle extends from the body to collect tissue samples using the cutter; and (b) a targeting set, wherein the targeting set includes: (i) a cannula, (ii) an obturator having an elongate shaft and a sharp distal tip, and (iii) an obturator actuation assembly, wherein the cannula defines a lumen, wherein the lumen is configured to separately receive the needle of the biopsy device and the obturator, wherein the obturator actuation assembly is configured to fire the obturator distally relative to the cannula to project the obturator out of the cannula and through tissue.

### EXAMPLE 37

The biopsy system of Example 36, wherein the obturator actuation assembly is configured to drive the obturator between an initial position and a cocked position, wherein the obturator actuation assembly is further configured to selectively maintain the obturator in the cocked position.

### EXAMPLE 38

The biopsy system of Example 36, wherein the obturator actuation assembly comprises a housing, a latch mechanism and a release mechanism, wherein the latch mechanism is secured to the obturator to selectively engage at least a portion of the housing.

### EXAMPLE 39

The biopsy system of Example 38, wherein the release mechanism is configured to selectively disengage the latch mechanism from the housing.

### EXAMPLE 40

A method for placing a MRI guided breast biopsy targeting set with firing obturator for use with positioning a biopsy device within a patient at a biopsy site, wherein the targeting set includes a targeting cannula defining a lumen extending longitudinally therethrough, an obturator and an obturator actuation assembly, wherein the method comprises: (a) drawing the obturator proximally relative to the obturator actuation assembly to place the obturator and the obturator actuation assembly in a cocked configuration; (b) inserting the obturator into the lumen of the targeting cannula; (c) inserting the obturator into tissue of a patient; (d) advancing the obturator to a first position, wherein the first position is within a predetermined distance of the biopsy site; (e) firing the obturator distally under the direction of the obturator actuation assembly, wherein firing the obturator distally advances the obturator relative to the biopsy site; (f) removing the obturator from the lumen of the targeting cannula; and (g) inserting a needle of a biopsy device into the lumen of the targeting cannula, thereby replacing the obturator with the needle of the biopsy device.

It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

We claim:

1. An MRI guided breast biopsy targeting set with firing obturator for use with positioning a biopsy device within a patient, the targeting set comprising:
(a) an obturator, wherein the obturator is insertable into a targeting cannula in lieu of a needle of a biopsy device; and
(b) a firing assembly, wherein the firing assembly includes:
(i) a housing,
(ii) a latch, and
(iii) a release, wherein the firing assembly is responsive to the release to disengage the latch and fire the obturator distally relative to the housing.

2. The targeting set of claim 1, wherein the obturator extends longitudinally through the housing of the tiring assembly.

3. The targeting set of claim 2, wherein the obturator comprises a pair of laterally extending arms, wherein the pair of arms are configured to translate the obturator proximally from an initial position to a cocked position.

4. The targeting set of claim 3, wherein translation of the obturator to the cocked position is configured to engage the latch mechanism.

5. The targeting set of claim 4, wherein the latch is configured to selectively maintain the obturator in the cocked position.

6. The targeting set of claim 3, wherein the laterally extending arms are bowed distally.

7. The targeting set of claim 3, wherein each arm of the laterally extending arms define grip rings.

8. The targeting set of claim 1, further comprising a cannula assembly, the cannula assembly comprising a hub and a cannula extending distally from the hub.

9. The targeting set of claim 8, wherein the cannula defines a lumen extending longitudinally from an open distal tip through the hub, wherein the lumen is configured to receive the obturator.

10. The targeting set of claim 9, wherein the obturator is movable relative to the cannula to retract the obturator within the lumen of the cannula.

11. The targeting set of claim 9, wherein the cannula comprises a first lateral aperture, wherein the obturator comprises a second lateral aperture, wherein the first and second lateral apertures are configured to align to receive tissue therein when the obturator is in an uncocked position.

12. The targeting set of claim 1, wherein the obturator comprises a sharp distal tip.

13. The targeting set of claim 1, wherein the firing assembly further comprises a resilient member, wherein the resilient member is configured to drive the obturator distally.

14. The targeting set of claim 13, wherein the resilient member comprises a coil spring.

15. The targeting set of claim 14, wherein the coil spring is oriented coaxially with the obturator.

16. An MRI guided breast biopsy system, comprising:
(a) a biopsy device, wherein the biopsy device includes:
(i) a body,
(ii) a needle, and
(iii) a cutter, wherein the needle extends from the body to collect tissue samples using the cutter; and
(b) a targeting set, wherein the targeting set includes
(i) a cannula,
(ii) an obturator having an elongate shaft and a sharp distal tip, and
(iii) an obturator actuation assembly, wherein the cannula defines a lumen, wherein the lumen is configured to separately receive the needle of the biopsy device and the obturator, wherein the obturator actuation assembly is configured to fire the obturator distally relative to the cannula to project the obturator out of the cannula and through tissue.

17. The biopsy system of claim 16, wherein the obturator actuation assembly is configured to drive the obturator between an initial position and a cocked position, wherein the obturator actuation assembly is further configured to selectively maintain the obturator in the cocked position.

18. The biopsy system of claim 16, wherein the obturator actuation assembly comprises a housing, a latch mechanism and a release mechanism, wherein the latch mechanism is secured to the obturator to selectively engage at least a portion of the housing.

19. The biopsy system of claim 18, wherein the release mechanism is configured to selectively disengage the latch mechanism from the housing.

20. A method for placing a MRI guided breast biopsy targeting set with firing obturator for use with positioning a biopsy device within a patient at a biopsy site, wherein the targeting set includes a targeting cannula defining a lumen extending longitudinally therethrough, an obturator and an obturator actuation assembly, wherein the method comprises:
(a) drawing the obturator proximally relative to the obturator actuation assembly to place the obturator and the obturator actuation assembly in a cocked configuration;
(b) inserting the obturator into the lumen of the targeting cannula;
(c) inserting the obturator into tissue of a patient;
(d) advancing the obturator to a first position, wherein the first position is within a predetermined distance of the biopsy site;
(e) firing the obturator distally under the direction of the obturator actuation assembly, wherein firing the obturator distally advances the obturator relative to the biopsy site;
(f) removing the obturator from the lumen of the targeting cannula; and
(g) inserting a needle of a biopsy device into the lumen of the targeting cannula, thereby replacing the obturator with the needle of the biopsy device.
